(19) United States
(12) Patent Application Publication
(10) Pub. No.: US 2006/0013876 A1
(43) Pub. Date: Jan. 19, 2006

(54) Novel floating dosage form

(76) Inventors: Braj Bhushan Lohray, Gujarat (IN); Sandip B. Tiwari, Gujarat (IN); Raveendra M Pai, Gujarat (IN); Krishna T. Murthy, Gujarat (IN); Pavak R. Mehta, Gujarat (IN)

Correspondence Address: NIXON & VANDERHYE, PC, 901 NORTH GLEBE ROAD, 11TH FLOOR, ARLINGTON, VA, 22203, US

(21) Appl. No.: 10/518,909

(22) PCT Filed: Jun. 25, 2003
(86) PCT No.: PCT/IN03/00229

§ 371 Date: Sep. 1, 2005

(30) Foreign Application Priority Data
Jun. 26, 2002 (IN) 565/MUM/2002

(51) Int. Cl. A61K 9/24

(52) U.S. Cl. 424/472

(57) ABSTRACT

Present invention relates to a novel pharmaceutical composition containing an active ingredient(s) which is retained in the stomach or upper part of gastrointestinal tract for controlled delivery of medicament for improved local treatment, and/or better absorption from upper parts of gastrointestinal tract for effective therapeutic results. Present invention also provides a method for preparation of the said dosage form preferably in the form of a bilayer tablet, in which one layer constitutes for spatial control and the other being for temporal control.

### FIELD OF INVENTION

Present invention relates to a novel pharmaceutical composition containing an active ingredient(s) which is retained in the stomach or upper part of gastrointestinal tract for controlled delivery of medicament for improved local treatment, and/or better absorption from upper parts of gastrointestinal tract for effective therapeutic results. Present invention also provides a method for preparation of the said dosage form preferably in the form of a bilayer tablet, in which one layer constitutes for spatial control and the other being for temporal control.

### BACKGROUND OF THE INVENTION

Oral administration of a drug is perhaps the least predictable route of drug administration, yet it is the route that is used most frequently. Oral medications such as tablets, capsules etc. are relatively cheap to manufacture, offer convenient form of drug administration and reduce the possibility of errors in total dose if the patient is self administrating the dosage form. Classically, oral medications are administered as immediate release dosage forms. The major disadvantage of such immediate release preparations is the repeated frequency of drug administration and fluctuations in drug plasma levels. Use of oral controlled release preparations circumvents these problems. Such type of drug delivery systems are designed to deliver the drug in such a way that the drug level is maintained within the therapeutic window and effective and safe blood levels are maintained for a period as long as the system continues to deliver the drug at a particular rate. Controlled drug delivery usually results in substantially constant blood levels of the active ingredient as compared to fluctuations observed with immediate release dosage forms. Controlled drug delivery results in optimum therapy, and not only reduces the frequency of dosing, but may also reduce the severity of side effects.

There are numerous advantages associated with the use of controlled drug delivery systems. The main benefit in controlled drug therapy is the improvement in efficiency of the treatment. Controlled drug therapy reduces the required frequency of administration, and single doses at periodic intervals are sufficient, resulting in improved patient compliance.

A variety of controlled release dosage form designs has been reported in literature. These controlled drug delivery systems are based on different modes of operation and have been variously named, for example, as dissolution controlled systems, diffusion controlled systems, ion exchange resins, osmotically controlled systems, erodable matrix systems, pH independent formulations, swelling controlled systems and the like.

An ideal controlled drug delivery system should deliver the drug at a constant rate as the system passes through the gastrointestinal tract. In practice however it is bit difficult. An orally administered drug delivery system encounters a wide range of highly variable conditions such as pH, agitation intensity, and composition of the gastrointestinal fluids as it passes down the gastrointestinal tract. Various researchers have attempted to design oral controlled drug delivery systems that overcome these problems and deliver the drug at a constant rate as it passes down the gastro-intestinal tract.

The absorption of the drug candidate from the gastrointestinal tract is dictated by the location of the dosage form in the gastrointestinal tract and the GI contents. Some drugs are more efficiently absorbed from the upper part of GI tract while others are absorbed from the lower parts of the gastro-intestinal tract. Therefore, in instances where the drug is not absorbed uniformly over the gastro-intestinal tract, the rate of drug absorption may not be constant in spite of the drug delivery system delivering the drug at a constant rate into the gastro-intestinal fluids. In such cases where the drug has a particular absorption site in the gastro-intestinal tract (i.e. absorption window), stomach or upper part of the small intestine for example, the drug may not be completely absorbed when administered in the form of a typical controlled drug delivery system. It is clear that for such drugs having an “absorption window” as stomach or upper parts of small intestine, an effective oral controlled drug delivery system should be designed not only to deliver the drug at a controlled rate, but also to retain the drug in the upper parts of the gastrointestinal tract for a long period of time.

WO 00/38650 describes composition for a pharmaceutical dosage form for prolonged release of an active agent from a multilayered dosage form having a highly swellable layer and a drug layer, the dosage form being adapted for retention in the stomach for a prolonged period. The dosage form upon contact with the aqueous fluid/gastric contents swells to a maximum extent leading to increased buoyancy of the dosage form and the whole dosage form will float on the surface of the gastric contents.

U.S. Pat. No. 6,207,197 assigned to West Pharmaceutical Services for Drug Delivery and Clinical Research Centre provides a drug delivery composition for the controlled release of an active agent in the stomach environment over a prolonged period of time. This comprises of microspheres comprising an active ingredient in the inner core of the microspheres and i) a rate controlling layer of a water insoluble polymer and ii) an outer layer of a bioadhesive agent in the form of a cationic polymer.

U.S. Pat. No. 5,780,057 assigned to Jagotec A G provides a pharmaceutical dosage form for oral administration is a two- or three-layered tablet, wherein at least one layer can rapidly swell by contact with biological and/or aqueous fluids. The said swelling results in a considerable increase in the tablet volume. This phenomenon determines a prolonged residence of the pharmaceutical form in the gastric mucosa and therefore allows slow release of the active ingredient from said pharmaceutical dosage form to the stomach and/or the upper part of the intestine.

U.S. Pat. No. 4,996,058 assigned to Ciba-Geigy corporation relates to a covered, solid retard form which in the case of oral administration remains in the stomach during periodic emptying and ensures continuous release. This dosage form contains at least one component that expands on contact with body fluid and contains a physiologically active substance or a combination of such substances. One permeable hydrophilic membrane which surrounds component which is expansible at the site of use and optionally a covering which surrounds components which, disintegrates without delay under the action of body fluid at the site of use.

JP 06024959 assigned to Bayer Yakuhin KK discloses a pharmaceutical composition to deliver the ciprofloxacin over a prolonged period of time by making the tablet to suspend in the stomach. The system contained two parts laminated to each other. One part containing a water-swellable gel-forming polymer and a water expandable foaming agent dispersed in the polymer and the other part containing the active agent. The results showed that only 46% of the drug was dissolved even after 24 hours loosing its practicability to be effective as once daily ciprofloxacin formulation.

U.S. Pat. Nos. 4,767,627, 4,735,804 and 4,758,436 present dosage forms of various geometry; continuous solid stick; tetrahedron; planar disc; multi-lobed flat device; and ring. The devices are compressible to a size suitable for swallowing, and are self-expandable to a size which prevents passage through the pylorus. They are sufficiently resistant to forces of the stomach to prevent rapid passage through the pylorus for a pre-determined period of time and erode in the presence of gastric juices. The devices are homogenous; they contain the same polymer constituents in different areas of the device. The tetrahedron shape presented in U.S. Pat. No. 4,735,804 is homogenous in its four lobes, which are attached to each other by a polymeric matrix.

The medicaments are incorporated into the device as a liquid solution or suspension, which may necessitate the addition of mentioned preservatives or buffering agents. Alternatively, the controlled release drug module may be tethered or glued to the device.

WO 01/64183 assigned to Ranbaxy Laboratories describes a pharmaceutical composition in the form of tablets or capsules which provides a combination of spatial and temporal control of the drug delivery, specifically for the drug ciprofloxacin. According to the invention, the pharmaceutical composition is prepared by mixing the drug with the gas generating component, the swelling agent, and one or both of the viscolysing agent and the gelling agent, plus other excipients and lubricants. The blend was either directly compressed into tablets or may be filled into capsules. Alternatively, the pharmaceutical composition is prepared by mixing the foregoing ingredients with only one-half of the lubricants. The blend is subjected to dry granulation technique by passing it through the roller compactor and then sieved to obtain granules. The granules are then mixed with the remaining lubricants, and filled into capsules or compressed into tablets. The floating in the stomach is achieved by interaction of the gas-generating component with the gastric hydrochloric acid resulting in gas-entrapped gel matrix having low density.

U.S. Pat. No. 3,574,820 describes the use of a gelatin matrix that hydrates in the stomach, gels, swells and cross-links with N-acetyl-homocysteine thiolactone to form a matrix too large to pass through the pylorus.

U.S. Pat. No. 4,207,890 discloses a drug dispensing device which comprises a collapsed, expandable imperforate envelope, made of a non-hydratable, body fluid and drug-permeable polymeric film, which contains the drug, and an expanding agent also contained within the polymeric envelope which, when in contact with body fluids, causes the envelope to expand to a volume such that the device is retained in the stomach.

U.S. Pat. No. 4,434,153 describes a device comprised of a matrix formed of a hydrogel that absorbs and imbibes fluid from the stomach, expands and swells, in order to retain in the stomach for an extended period of time, and a plurality of tiny pills dispersed throughout the matrix, having a drug-containing core and a fatty acid and wax wall surrounding the core. A significant disadvantage of the devices of the above publications is that they appear to ignore natural contractions of the stomach which may contribute to a rapid diminishing of size, leading to early removal of the device from the stomach. These devices lack the mechanical strength required to withstand the natural mechanical activity, that includes contractions of the stomach.

U.S. Pat. Nos. 5,002,772 and 5,443,843 disclose an oral drug delivery system having a delayed gastrointestinal transit, which releases the drug/s contained therein in a controlled manner and which in their expanded form resist gastrointestinal transit. These delivery systems comprise one or more retention arms as a non-continuous compressible element, and an attached controlled release drug-containing device. The preferred configuration is a coil or a spiral. These systems must comprise at least two distinct parts out of which at least one is retention arm and a controlled release arm.

U.S. Pat. Nos. 5,047,464 and 5,217,712 describe a system comprising bio-erodible, thermoset, covalently cross-linked, poly (ortho) ester polymers, which expand from a compressed state upon delivery thereof. The acidic environment of the stomach eventually results in the degradation of the polymers within the system, thus permitting its removal from the stomach. The system is characterized by high resiliency.

U.S. Pat. No. 5,651,985 describes a system devised from a mixture of polyvinyl-lactams and polyacrylates which are characterized by their high degree of swelling in the stomach resulting in its retention in the stomach for a prolonged period of time.

U.S. patent publication No. 20030021845 describes a gastroretentive drug delivery system comprising a single- or multi-layered matrix comprising a polymer selected from degradable polymers that may be hydrophilic polymers not instantly soluble in gastric fluids, enteric polymers substantially insoluble at pH less than 5.5 and/or hydrophobic polymers and mixtures thereof; non-degradable polymers; and any mixtures thereof, a continuous or non-continuous membrane comprising at least one polymer having a substantial mechanical strength; and a drug; wherein the matrix when affixed or attached to the membrane prevents evacuation from the stomach of the delivery system for a period of time of from about 3 to about 24 hours.

Not withstanding the above referenced prior information, the present inventors have developed more simpler and possibly more convenient dosage form preferably in the form of a bilayer tablet or caplet, in which one layer constitutes for spatial control and the other being for temporal control.

The present invention involves delivering the drug in the form of a bilayer dosage form in which one layer constitutes for spatial control and the other being for temporal control.

Spatial control layer comprises of low bulk density polymers such as cellulosic derivatives either natural, synthetic or semi-synthetic, ethyl cellulose in particular, polyethylene oxide, fatty acids, hydrogenated oils, waxes, shellac, and the likes either alone or in combination. Other optional pharmaceutical excipients may also be incorporated. The temporal control layer comprises of controlled release matrix polymers such as synthetic or semisynthetic cellulose derivatives like hydroxypropyl methylcellulose, ethylcellulose and the like and/or natural polymers or gums such as xanthan gum, gelatin and the like, acrylic acid derivatives, polyvinyl acetate along with other optional pharmaceutical excipients. The active pharmaceutical ingredient is incorporated into the temporal control layer. The temporal control layer may also contribute to floating of the dosage form once the system absorbs aqueous fluids from GI tract leading to swelling and decrease in density. The final dosage form may be coated with suitable coating materials for either functional or non-functional use known to those in the art of formulation development.

### OBJECTIVES OF THE PRESENT INVENTION

The objective of the present invention is to provide a novel gastro-retentive delivery system for controlled release of therapeutically active agent in stomach or upper part of gastro-intestinal tract in the form of bilayer dosage form in which;
One layer (Layer-A) is responsible for retaining the dosage form in stomach or upper part of gastro-intestinal tract (spatial control) for prolonged period and is composed of pharmaceutical excipients with low bulk density such as cellulosic derivatives either natural, semi-synthetic or synthetic, ethyl cellulose in particular, polyethylene oxide, fatty acids, hydrogenated oils, waxes, shellac, and the likes either alone or in combination and along with other optional pharmaceutical excipients.
The second layer (Layer-B) is responsible for prolonged or controlled drug delivery (temporal control) and comprises of controlled release matrix polymers such as synthetic or semisynthetic cellulose derivatives like hydroxypropyl methylcellulose, ethylcellulose and the like and/or natural polymers or gums such as xanthan gum, gelatin, acrylic acid derivatives, polyvinyl acetate and the like along with other optional pharmaceutical excipients.

The dosage forms of the present invention can be a tablet or caplet either coated or uncoated, or tablet filled in capsules.

Another objective of the present invention is to provide a novel gastro-retentive delivery system for controlled release of therapeutically active agent having absorption window and/or site of action as stomach or upper parts of gastro-intestinal tract for prophylactic and therapeutic use.

Still another objective of the present invention is to make the dosage form float on the surface of the gastric contents with controlled release of the active agent wherein the drug is delivered over a period of time which is equal to or less than the transit time of the dosage form in the absorptive region of the gastrointestinal tract.

A further objective of the present invention is to release the active pharmaceutical agent having absorption window as stomach or upper part of gastro-intestinal tract in a slow, controlled manner for better absorption and better efficacy compared to other conventional and controlled release dosage forms.

Yet another objective of the present invention is to provide a drug delivery system that can incorporate high and low dose medicament without compromising dosage form characteristics/properties with acceptable size for oral administration.

### DETAILED DESCRIPTION OF INVENTION

The present invention relates to a novel pharmaceutical technology in the form of bilayer buoyant matrix dosage form to prolong the delivery of the drug in the stomach or upper part of small intestine. One layer makes the dosage form to stay/float on the surface of the contents in the stomach giving spatial control and the other layer containing the drug and controlled release matrixing polymers optionally along with the other pharmaceutical ingredients for temporal control of the drug.

According to the present invention, the novel technology aims to retain the pharmaceutical dosage form in the stomach or upper part of small intestine. This is achieved through a bilayered pharmaceutical composition wherein one layer (Layer-A) is responsible for spatial control by making the whole dosage form to float on the surface of the aqueous/gastric contents of the gastrointestinal tract and the other layer (Layer-B) comprising of active pharmaceutical ingredient and controlled release matrixing polymers along with optional pharmaceutical excipients, thereby allowing prolonged release of the drug candidate.

In the present invention, the layer A which is responsible for the buoyancy of the whole dosage form is composed of polymers and/or suitable excipients whose density is below one because of which the dosage form floats along with the other layer on the surface of the gastric and/or aqueous media. In the present invention the polymers used in the layer-A can be various cellulosic derivatives either synthetic or semisynthetic, whose density is less than one, preferably ethylcellulose, either alone or in combination with pharmaceutical ingredients like, hydrogenated oils, waxes, fatty acids, shellac, polyethylene-oxide and the likes.

According to present invention the ratio between ethylcellulose and hydrogenated oils for Layer A can vary from 95:5 to 30:70.

According to the present invention, the layer B contains the active pharmaceutical ingredient along with, rate retarding polymers, which may optionally be combined with fillers, binders, superdisintegrating agents and other pharmaceutically acceptable lubricants, glidants or anti adherents. The layer-B can be prepared using various release rate retarding polymers such as cellulose derivatives synthetic or semisynthetic like hydroxypropyl methylcellulose, hydroxy ethylcellulose and the like and/or natural polymers or gums such as xanthan gum, gelatin and/or polyethylene oxide or other synthetic polymers such as acrylic acid derivatives, polyvinyl acetate and the likes along with other optional pharmaceutical excipients. The pharmaceutical composition may be optionally coated with agents as is known in the art.

According to the present invention the drugs can belong to any class and for any disorder by which the therapy or chemotherapy would be improved as a result of controlled drug delivery. The drug may be pharmacologically or chemotherapeutically active itself, or may be converted into active species by a chemical or enzymatic process in the body. Examples of suitable drugs candidates and drugs used for different disorders, are antibiotics, anti-cancers, anti-fungals, anti-fibrial and antiviral agents, lipid lowering agents, non-steroidal anti-inflammatory agents, anti-ulcer agents, drugs for respiratory therapy, dopaminergic agents, skeletal muscle relaxants, cardiovascular agents, anti-epileptic, immunosupressants, anti-gout, antipsychotics. Preferable drugs from these classes are those whose absorption window and or site of action is stomach or upper part of the small intestine and also drugs which do not show uniform absorption characteristics throughout the gastro-intestinal tract.

Illustrative examples of drugs that are suitable for the present invention include antibacterial/anti-infective agents, such as ofloxacin, ciprofloxacin, cefuroxime, cefatrizine, cefpodoxime, clarithromycin, loracarbef, azithromycin, cefadroxil, cefixime, amoxycillin and the like; antivirals, such as acyclovir; cardiovascular agents, such as diltiazem, captopril, and the like; lipid lowering agents such as simvastatin, lovastatin, atorvastatin, and the like; non-steroidal anti-inflammatory agents such as etodolac, ketorolac, and the like; anti-ulcer agents, such as ranitidine, famotidine and the like; drugs for respiratory diseases, such as fexofenadine, pseudoephedrine, phenylpropanolamine, dextromethorphan, chlorpheniramine, and the like; dopaminergic agents, such as bromocriptine; immunosuppressants, such as cyclosporin; skeletal muscle relaxants, such as baclofen: anti-gout agents, such as allopurinol: and the like: antidiabetic agents such as acarbose, glipizide and the like. The drug itself or its pharmaceutically acceptable salt or ester may be used in the present invention. Moreover combinations of drugs that are typically administered together may be included as the drug component of the pharmaceutical composition. The amount of drug to be used in the composition is that which is typically administered for a given period of time. The drugs can be present in the composition of about 0.2 to 1000 mg depending on the drug candidate.

The release-retarding polymers used in the invention belong to the class of cellulosics natural gums and/or acrylic acid derivatives which may be either hydrophobic or hydrophilic. Release retarding polymers may be selected from hydroxypropyl methylcellulose (HPMC), hydroxypropylcellulose (HPC), sodium carboxymethylcellulose (Sodium CMC), ethylcellulose, xanthan gum, guar gum, acrylic acid derivatives, polyvinyl acetate, polyethylene oxide and the likes known to those in the art. The release retarding agents may be added in the range of 0.5 to 50% of total weight of the composition of layer B

Disintegrants when used in the pharmaceutical composition swells upon contact with the aqueous media and burst release of the drug is observed. Disintegrating agents used in the present composition maybe selected cross-linked polyvinyl pyrrolidone, sodium starch glycolate or cross-linked sodium carboxy methylcellulose, microcrystalline cellulose, starch, pregelatined starch and the likes, preferably cross-linked sodium carboxymethylcellulose is used. The disintegrating agent may be present in an amount from 0.1 to 20%, preferably from 0.2 to 10% and more preferably from 0.5 to 5%, by weight of the total weight of the composition of layer B (w/w).

Pharmaceutical lubricants used in the present invention maybe selected from stearic acid, magnesium streate, zinc stearate and the like, silicone dioxide, hydrogenated vegetable oils, glyceryl behenate, glyceryl monosterate, talc and the like. In the present invention, the amount of lubricant used may be in the range from about 0.1 to 5% and more preferably in the range of 0.1 to 3% by weight of the total weight of the composition.

The binders used is selected from natural polymers selected from starch or gum including acacia, tragacanth, gelatin or synthetic polymers selected from polyvinyl pyrrolidone, methylcellulose, ethylcellulose, hydroxypropyl methylcellulose, sodium carboxymethylcellulose, hydroxypropyl cellulose.

The filler used in the present invention may be lactose, mannitol, starch, pregelatinized starch, cellulose and the likes. The concentration of the fillers in the present invention may vary from about 2 to 80% of the total weight of the composition of the layer B.

The pharmaceutical dosage form upon oral administration floats on the surface of the gastric content based on the principle of buoyancy due to hydrodynamically balancing property of the low density polymers (Layer-A) and releases the drug in a controlled fashion from the other layer (layer B) by diffusion and/or erosion mechanism for a prolonged period.

Process for Preparation

According to the present invention the pharmaceutical dosage form contains two layers, one responsible for the buoyancy (Layer A) and other being the drug layer in the form of a matrix system (Layer B). In the present invention, the granules for the two different layers are prepared separately and then compressed into bilayered tablet or caplet with suitable punch using suitable tablet compression machine.

According to the present invention, the buoyant layer (Layer A) can be prepared by physical mixing of the suitable excipients mentioned above and can be compressed directly along with Layer-B. Alternatively, the granules can also be prepared by hot melt granulation or wet granulation technique using the suitable mixture of above mentioned ingredients to obtain suitable size granules.

According to the present invention, the granules of the drug layer (Layer B) can be prepared by direct compression, dry granulation or by wet granulation technique. In case of wet granulation, the drug with release retarding polymers, optionally with fillers, disintegrating agent was converted into dough mass using binder in a particular solvent. The mass was sieved and dried. The dried mass was sieved through ASTM #20. These granules are then mixed with the lubricants and compressed into tablets along with granules of Layer A.

Coating Process

The present invention relating to a buoyant pharmaceutical composition and a method to prepare it in the form of tablets as described above may be optionally coated with rapidly dissolving water-soluble pharmaceutical excipients to mask the bitter taste of the drug and/or to protect the dosage form from degradation during varying storage conditions. A coating of low viscosity hydrophilic polymer is preferred for the faster hydration and release of the drug.

The film former can be cellulose derivatives including hydroxypropyl methylcellulose, ethylcellulose and the like. Highly water-soluble pharmaceutical excipients can be included in the coating to support the faster dissolution of the polymer. The water-soluble ingredient includes lactose, sucrose and the like. The solvent used for the coating solution in the present invention may be water, isopropyl alcohol or methylene chloride and mixture of the same. The tablet may be coated to a weight gain of 0.5% to 8%, preferably 1% to 5%.

Following non-limiting example describe the illustrative pharmaceutical compositions of the present invention and the means of carrying out the invention to obtain a pharmaceutical dosage form of various active agents for oral controlled release.

### EXAMPLE-1

TABLE 1

| Ingredients | Mg/tab |
|---|---|
| Layer-A | |
| Ethyl cellulose | 172 |
| Hydrogenated castor oil | 116 |
| Mg. Stearate | 6 |
| Talc | 6 |
| Total weight | 300 |
| Layer B | |
| Ofloxacin | 800 |
| HPMC-K15 | 55.5 |
| Cross-linked sod CMC | 23 |
| PVP-K 90 | 27 |
| Isopropyl alcohol | q.s. |
| Magnesium stearate | 9.25 |
| Talc | 9.25 |
| Total weight | 1224 |

Layer A

Ethylcellulose and hydrogenated castor oil are mixed together and the blend was heated on a controlled temperature water bath at 90° C. to obtain a congealed mass. The congealed mass was cooled to room temperature and sieved through ASTM sieve 20. The blend was then lubricated.

Layer B

All the ingredients used in the formulation were passed through a sieve (ASTM # 60). Ofloxacin, HPMC and cross-linked sodium carboxymethylcellulose were mixed together with polyvinylpyrrolidone (PVP) as a binder. The mass was dried and passed through a sieve (ASTM #20). Tablets were prepared using granules of Layer A and Layer B using a rotary bilayer tablet compression machine using suitable punch. The tablets were spray coated with hydroxypropyl methylcellulose (HPMC, 6 cps) to obtain the weight gain in the range of 2-4%.

Dissolution study of the coated tablets was conducted in 0.1N HCl using USP Apparatus 1 (basket) at 100 rpm. The dissolution results are given in Table-2

TABLE 2

| Time (hr) | % drug release |
|---|---|
| 1 | 33.0 |
| 2 | 57.2 |
| 3 | 73.19 |
| 4 | 90.03 |
| 5 | 96.5 |

### EXAMPLE-2

TABLE 3

| Ingredients | Mg/tab |
|---|---|
| Layer-A | |
| Ethyl cellulose | 90 |
| Hydrogenated castor oil | 60 |
| Mg. Stearate | 3 |
| Talc | 3 |
| Total weight | 156 |
| Layer B | |
| Ciprofloxacin | 500 |
| HPMC-K15 | 40 |
| Cross-linked sodium CMC | 15 |
| PVP-K 90 | 18 |
| Isopropyl alcohol | q.s. |
| Magnesium stearate | 4 |
| Talc | 4 |
| Total weight | 737 |

Layer A

Ethylcellulose and hydrogenated castor oil are mixed together and the blend was lubricated.

Layer B

All the ingredients used in the formulation were passed through a sieve (ASTM # 60). Ciprofloxacin, HPMC and cross-linked sodium carboxymethylcellulose were mixed together with polyvinylpyrrolidone (PVP) as a binder. The mass was dried and passed through a sieve (ASTM #20). Tablets were prepared using rotary bilayer tablet compression machine with suitable punch. The tablets were spray coated to obtain the weight gain in the range of 2-4%.

Dissolution study of the coated tablets was conducted in 0.1N HCl using USP Apparatus 1 (basket) at 100 rpm. The dissolution results are given in Table-4

TABLE 4

| Time (hr) | % drug release |
|---|---|
| 1 | 41.50 |
| 2 | 60.48 |
| 4 | 85.01 |
| 6 | 96.246 |

### EXAMPLE-3

TABLE 5

| Ingredients | Mg/tab |
|---|---|
| Layer-A | |
| Ethyl cellulose | 85 |
| Hydrogenated castor oil | 45 |
| Mg. Stearate | 3 |
| Talc | 3 |
| Total weight | 136 |
| Layer B | |
| Acyclovir | 525 |
| HPMC-K15 | 60 |
| Cross-linked sod CMC | 80 |
| PVP-K 90 | 8 |
| Isopropyl alcohol | q.s. |
| Magnesium stearate | 8 |
| Talc | 7 |
| Total weight | 824 |

Layer A

Ethylcellulose and hydrogenated castor oil are mixed together and the blend was lubricated.

Layer B

All the ingredients used in the formulation were passed through a sieve (ASTM # 60). Acyclovir, HPMC and cross-linked sodium carboxy methylcellulose were mixed together with polyvinylpyrrolidone (PVP) as a binder. The mass was dried and passed through a sieve (ASTM # 20). Tablets were prepared using rotary bilayer tablet compression machine with suitable punch. The tablets were spray coated to obtain the weight gain in the range of 2-4%.

Dissolution study of the coated tablets was conducted in 0.1N HCl using USP Apparatus 1 (basket) at 100 rpm. The dissolution results are given in Table-6

TABLE 6

| Time (hr) | % drug release |
|---|---|
| 1 | 24.0 |
| 2 | 33.0 |
| 4 | 43.02 |
| 6 | 55.82 |

Advantages of the present invention:
1. The present invention provides combined benefit of providing spatial control (targeted drug release) and temporal control (prolonged drug release)
2. There is no lag time for floating of the composition prepared according to the present invention.
3. The present invention does not require the use of gas generating components for providing the floating characteristics.
4. The composition of the present invention can be administered to the patients suffering from achlorhydria.
5. The operational simplicity and cost effectiveness of the present invention makes it suitable for industrial application.
6. It is possible to modify the floating characteristics by manipulating the shape and size of the dosage form.

1. A pharmaceutical gastro-retentive delivery system for controlled release of therapeutically active agent in stomach or upper part of gastrointestinal tract in the form of bilayer dosage form which comprises,
a) a first layer (layer-A) which is responsible for retaining the dosage form in stomach or upper part of gastrointestinal tract (spatial control) for a prolonged period, comprising of pharmaceutical excipients having low bulk density, selected from a mixture consisting of
(i) polymers selected from ethylcellulose or suitable enteric polymers of cellulose derivatives and
(ii) hydrogenated oils, waxes, fatty acids either alone or in combination; optionally with other pharmaceutical aids;
b) a second layer (Layer-B) which is responsible for prolonged or controlled drug delivery (temporal control) of therapeutic agent which comprises of the active agent and controlled release matrix polymers optionally with other pharmaceutical aids.

2. The delivery system as claimed in claim 1 wherein said pharmaceutical excipient with low bulk density is ethyl cellulose in combination with hydrogenated oils.

3. The delivery system as claimed in claim 1 wherein the ratio of ethylcellulose and hydrogenated oils is in the range of 95:5 to 30:70.

4. The delivery system as claimed in claim 1 wherein said pharmaceutical aids are selected from pharmaceutical lubricants, antiadherents and glidants.

5. The delivery system as claimed in claim 4, wherein said pharmaceutical aids are selected from magnesium stearate, talc, colloidal silicon dioxide, stearic acid, magnesium stearate flimerate, glyceryl behenate and hydrogenated oils or combination thereof

6. The delivery system as claimed in claim 1 wherein said controlled release matrix polymers is selected from synthetic or semisynthetic cellulose derivatives like hydroxypropyl methylcellulose, ethylcellulose, hydroxypropylcellulose, methylcellulose, sodium carboxymethylcellulose, natural polymers such as xanthan gum, gelatin, synthetic polymers, acrylic acid derivatives and polyvinyl acetate or mixtures thereof.

7. The delivery system as claimed in claim 1 wherein said pharmaceutical aids are selected from group of pharmaceutical fillers, disintegrants, lubricants, binders, antiadherents and glidants or combinations thereof.

8. The delivery system as claimed in claim 7 wherein said, pharmaceutical disintegrants are selected from crosslinked polyvinylpyrrolidone, crosslinked sodium carboxymethyl cellulose, sodium starch glycolate, microcrystalline cellulose, starch, and pregelatinized starch or their combinations.

9. The delivery system as claimed in claim 7 wherein said pharmaceutical binders are selected from natural polymers selected from starch or gum including acacia, tragacanth, gelatin or synthetic polymers selected from polyvinyl pyrrolidone, methyl cellulose, ethyl cellulose, hydroxypropyl methylcellulose, sodium carboxymethylcellulose, hydroxypropyl cellulose.

10. The delivery system as claimed in claim 7 wherein said pharmaceutical antiadherents, glidants and lubricants are selected from magnesium stearate, talc, colloidal silicon dioxide, stearic acid, salts of stearic acid, magnesium stearate flimarate, glyceryl behenate and hydrogenated oils.

11. The delivery system as claimed in claim 1, wherein said therapeutically active agent is in the form of a raw powder, dispersed or embedded in a suitable liquid, semisolid, micro- or nanoparticles, micro- or nanospheres, a tablet, a caplet, or in a suitable processable form.

12. The delivery system as claimed in claim 1, wherein said therapeutically active agent is a drug having a narrow absorption window in the gastrointestinal tract.

13. The delivery system as claimed in claim 1, wherein said therapeutically active agent is selected from the group consisting of therapeutic, chemotherapeutic, antibiotic antidiabetic, anti-cancers, anti-Fungals, anti-filarial, antiviral agents, lipid lowering agents, analgesics, non-steroidal anti-inflammatory agents, anti-ulcer agents, anti-epileptics, anti-gout, immunosuppressants, drugs for respiratory therapy, dopaminergic agents, skeletal muscle relaxants, cardiovascular agents, antipsychotics or those drugs which does not show uniform absorption characteristic throughout the length of the gastrointestinal tract.

14. The delivery system as claimed in claim 1, wherein said therapeutically active agent may also be a drug for local treatment of the gastrointestinal tract.

15. The delivery system as claimed in claim 1, wherein said therapeutically active agent is selected from antibacterial/anti-infective agents, such as ofloxacin, ciprofloxacin, cefiroxime, cefatrizine, cefpodoxime, clarithromycin, loracarbef, azithromycin, cefadroxil, cefixime, amoxycillin; antivirals, such as acyclovir; cardiovascular agents, such as diltiazem, captopril; lipid lowering agents such as si.mvastatin, lovastatin, atorvastatin; non-steroidal anti-inflammatory agents such as etodolac, ketorolac; anti-ulcer agents such as ranitidine, famotidine; drugs for respiratory diseases, such as fexofenadine, pseudoephedrine, phenylpropanolamine, dextromethorphan, chlorpheniramine; dopaminergic agents, such as bromocriptine; immunosuppressants, such as cyclosporin; skeletal muscle relaxants, such as baclofen; anti-gout agents, such as allopurinol; antidiabetic agents such as acarbose, glipizide.

16. Use of the delivery system as claimed in claim 1, for treatment of disease conditions as described in any preceding claims above.

17. The delivery system as claimed in claim 1 wherein the layers A & B are prepared by technique selected from melt granulation, wet granulation or direct compression.

18. The delivery system as claimed in claim 1 wherein the amount of therapeutically active agent is present in an amount ranging from about 0.2 to 1000 mg.

19. The delivery system as claimed in 1 wherein the dosage form floats on the surface of the gastric fluid for prolonged period ranging from 0.5 to 10 hours.

20. The delivery system as claimed in claim 1 which is optionally coated with rapidly dissolving water soluble film forming polymer or rapidly dissolving pharmaceutical excipient.

21. A drug delivery system as claimed in claim 1 which includes tablets, caplets or tablets filled in capsules.

22. A pharmaceutical composition prepared according to the present invention suitable for human administration.
